(19) United States
(12) Patent Application Publication
(10) Pub. No.: US 2004/0062743 A1
(43) Pub. Date: Apr. 1, 2004

(54) Acid-containing desensitization agents for teeth

(76) Inventors: Alexander Karl Huwig, Horgen (CH); Norbert Moszner, Eschen (LI); Frank Zeuner, Vaduz (LI); Volker Rheinberger, Vaduz (LI)

Correspondence Address: Nixon Peabody LLP, Clinton Square, P.O. Box 31051, Rochester, NY, 14603-1051, US

(21) Appl. No.: 10/658,953

(22) Filed: Sep. 9, 2003

(30) Foreign Application Priority Data
Sep. 27, 2002 (DE) 102 45 212.1

(51) Int. Cl. A61K 31/785

(52) U.S. Cl. 424/78.26

(57) ABSTRACT

Compositions which contain an acid and an organic polymer that has carboxyl and/or hydroxyl groups, and which are suitable in particular or the desensitization of teeth.

The invention relates to acid-containing compositions which are suitable in particular for the desensitization of teeth.

A hypersensitivity or hyperaesthesia of the teeth to physical (cold, contact, heat), chemical (acid) or osmotic irritations is mostly to be attributed to an exposure of the dentine, in particular in the area of the tooth necks and the root areas. A cause of this can be the disappearance of the protective tooth enamel as a result of erosion, abrasion or breaking off or through the exposure of the root surface due to receding gums or as a result of a periodontal treatment.

It is assumed that the pain is triggered by movement of the fluid (dentinal fluid) present in the tubules of the dentine. This results in a brief compression or expansion of the nerve cells which are present in the pulp and which react to this by triggering and transmitting a pain signal.

The creation of a pain signal in a nerve cell is linked to a potassium-ion gradient. In the resting state, the potassium-ion concentration inside the nerve cell is greater than outside. In the case of an irritation, ion channels are opened which allow the potassium ions to flow outwards and thus trigger the pain signal. By setting a high potassium-ion concentration on the outside of the nerve cell, the outflow of potassium ions from the cell and therefore the creation and transmission of the pain signal can be prevented. When treating hypersensitive teeth, however, only short-term successes are achieved by increasing the extracellular potassium-ion concentration, for example using potassium-ion-containing mouth-rinse solutions or toothpastes.

In addition to increasing the extracellular potassium-ion concentration, a desensitization of sensitive teeth can be achieved by sealing the dentinal tubules. This prevents fluid movements in the tubules and therefore irritations of the nerve cells.

Septodont Pharm-Dental Handelsgesellschaft m.b.H. sells a product under the name Isodan® which contains hydroxyethyl methacrylate (HEMA) in addition to potassium nitrate and sodium fluoride and is intended to prevent fluid movements inside the tubules through coagulation of the proteins of the tubules.

In the product Gluma® of Heraeus Kulzer, the protein-precipitating properties of HEMA are combined with the cross-linking properties of glutaraldehyde. Glutaraldehyde is intended to covalently bond the precipitated proteins of the tubules with the collagen contained in the dentine.

Taking health aspects into consideration, the use of glutaraldehyde in medicinal products is not harmless due to its alkylating effect. Furthermore, only an insufficient desensitization of teeth is achieved with the known materials even when using cross-linkers.

The object of the invention is to prepare desensitization agents for teeth that facilitate a long-lasting desensitization.

This object is achieved by compositions which, in addition to an acid, contain an organic polymer which has carboxyl and/or hydroxyl groups.

Polymers containing carboxyl groups are not acids within the meaning of the invention. According to the invention, by acids non-polymer compounds are meant. Organic acids, such as carboxylic acids, sulphonic acids and in particular phosphonic acids, are particularly suitable as acids.

According to the invention, acids which have a high solubility in water or in water/ethanol mixtures are preferred. By a high solubility is meant a solubility of 0.5 to 50 wt.-%, preferably 20 to 50 wt.-% in water or a mixture of 50 wt.-% water and 50 wt.-% ethanol.

Furthermore, acids which also have calcium-precipitating properties in addition to protein-precipitating properties are particularly preferred. Protein and calcium-precipitating properties are detected in standardized tests. Protein precipitation is measured as described in Example 6, the acids to be examined in the solution described in Example 5 (2^{nd} component) being present in a concentration of 0.2 M. The acid-containing solution is mixed with the protein solution described in Example 6 and the mass of the obtained precipitate is determined. According to the invention, acids which yield a pellet weight of >25 mg, preferably >30 mg and in particular >40 mg are preferred. The upper limit of the pellet weight is determined by the masses of the components used. Acids which yield a quantitative precipitation under the described conditions are particularly suitable according to the invention.

In order to determine the calcium-precipitating properties, a mixture of 5.0 g modified polyacrylic acid (PAA/GMA), 20 g polyethylene glycol (PEG 1000 DMA), 5.0 g hydroxypropyl cellulose, 0.3 g potassium fluoride and 65.6 g ethanol/water (50:50) with CaCl₂ is set at a Ca concentration of 0.1 M and 1 ml of this mixture is then reacted with 0.05 M of the acid to be tested. The acid-containing mixture is shaken vigorously, centrifuged in an Eppendorf centrifuge at 13,000 g for 5 minutes and dried until a constant weight is achieved. Acids which in this test yield a pellet weight of >30 mg, preferably ≧40 mg, particularly preferably 40 to 60 mg and in particular 40 to 50 mg are preferred. Polyacrylic acid with an average molecular weight of approximately 30,000 g/mol, which was modified by reaction with 0.5 mol glycidyl methacrylate (GMA) per acrylic-acid component in the polymer, is used as modified polyacrylic acid (PAA).

Preferred phosphonic acids are those according to the following Formula 1:

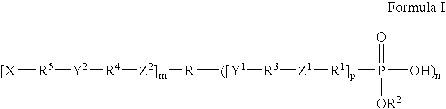

in which

n is 1, 2, 3 or 4,

m is 0, 1 or 2,

p is 0 or 1,

R is a straight-chained or branched aliphatic hydrocarbon radical with 1 to 12 carbon atoms or an aromatic hydrocarbon radical with 6 to 12 carbon atoms or an aliphatic/aromatic hydrocarbon radical with 7 to 16 carbon atoms, which can be substituted by OH, NH₂ and/or COOR⁶,

R¹ is a C₁ to C₁₂ alkylene, C₄ to C₁₂ cycloalkylene, C₆ to C₁₂ arylene or C₇ to C₁₆ alkylenearylene radical, which can be substituted by OH, NH₂ and/or COOR⁶, or is absent,

R² is H, a C₁ to C₆ alkyl or a phenyl radical,

R³, R⁴ each mean, independently of each other, a C₁ to C₁₂ alkylene, C₆ to C₁₂ arylene or C₇ to C₁₆ alkylenearylene radical, which can be substituted by methyl, phenyl or fluorine, or are absent,

R⁵ is —CH═CR¹³—, a prop-1-ene-1,3-diyl, C₁ to C₆ alkenylene, C₃ to C₉ cycloalkylene, C₁ to C₆ alkylene or phenylene radical or a group with the formula

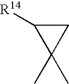

R⁶ is H, a C₁ to C₆ alkyl or a phenyl radical,

Z¹, Z² each mean, independently of each other, CO—O, CO—NR⁷, O—CO—NH, O, NH, S or are absent,

Y¹, Y² each mean, independently of each other, O, CO—O, CO—NR⁸, O—CO—NH or are absent,

R⁷, R⁸ each mean, independently of each other, H, or a C₁ to C₆ alkyl radical,

X is H, CN, N(R⁹)₂, OR¹⁰, COOR¹¹ or CONR₂¹²,

R⁹, R¹⁰, R¹¹, R¹² each mean, independently of each other, H, a C₁ to C₁₀ alkyl or a phenyl radical,

R¹³ is H or a methyl radical,

R¹⁴ is H or a C₁ to C₁₀ alkyl, vinyl or phenyl radical.

By alkylenearylene radicals is meant groups which contain both alkylene and arylene radicals, such as for example —CH₂-phenylene-CH₂—.

Preferred definitions of the variables of Formula 1 which can be chosen independently of each other are:

n is 1, 2 or 3, in particular 1 or 2 and/or

m is 1 and/or

p is 0 and/or

R is an aliphatic straight-chained or branched mono- to pentavalent alkane radical with 1 to 7 carbon atoms, an aromatic hydrocarbon radical with 6 carbon atoms or an aliphatic/aromatic hydrocarbon radical with 8 carbon atoms and/or

R¹ is a methylene or ethylene radical or is absent and/or

R² is H, a methyl or ethyl radical and/or

R³, R⁴ each mean, independently of each other, a methylene, ethylene, trimethylene, p-phenylene, ethylidene, 1-methylene ethane-1,2-diyl radical or are absent and/or

R⁵ is a methylene, ethylene, trimethylene, ethene-1,2-diyl, methylethylene, prop-1-ene-1,3-diyl, or a cyclopropylidene radical monosubstituted in 2 position or is absent, is in particular a methylene, ethylene or cyclopropylidene radical monosubstituted in 2 position or is absent and/or

R⁶ is H and/or

Z¹, Z² each mean, independently of each other, CO—O, O—CO—NH or O or are absent and/or

Y¹, Y² each mean, independently of each other, O, CO—O or CO—NR⁸ or are absent and/or

R⁷, R⁸ each mean, independently of each other, H or a methyl or ethyl radical and/or

X is H, CN, COOR¹¹ or CONR₂¹² and/or

R⁹, R¹⁰, R¹¹, R¹² each mean, independently of each other, H or a methyl, ethyl or phenyl radical and/or

R¹³ is H or a methyl radical,

R¹⁴ is H or a vinyl or phenyl radical.

Phosphonic acids, several and preferably all variables of which have one of the preferred definitions are particularly preferred.

Formula 1 includes all stereoisomers and their mixtures possible through the named substituents, such as racemates.

The phosphonic acids of Formula (1) can be prepared by synthesis of the corresponding phosphonic acid diesters and subsequent selective ester hydrolysis. Suitable alkyl phosphonic acid esters (APE) can be obtained in various ways. A proven method for the preparation of alkane phosphonic acid esters is the Michaelis Arbusow reaction (cf. G. M. Kosolapoff, Org. Reactions 6 (1951) 273), in which trialkylphosphites, e.g. triethylphosphite, and halogenoalkanes are reacted with each other, e.g.:

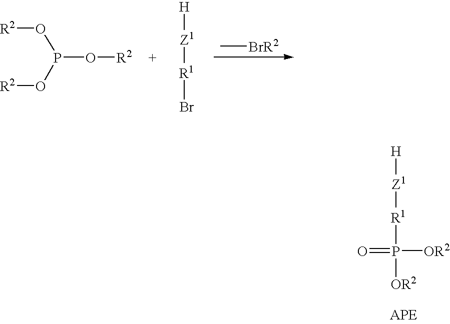

Concrete example:

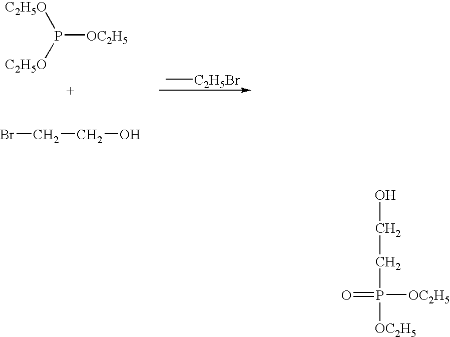

The substituent Z must be protected if necessary.

A further possibility for the synthesis of hydroxyalkylphosphonic acid esters (Z=OH) is the base-catalyzed addition of dialkylphosphites to mono- or difunctional aldehydes or ketones (analogous to: F. Texier-Boullet, A. Foucaud, Synthesis, 1982, 916):

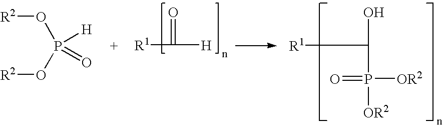

Concrete example:

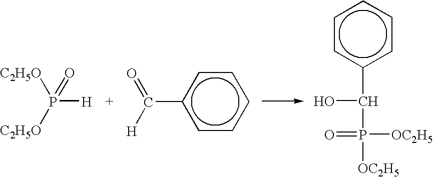

Acyloxyalkane phosphonic acid diesters can be obtained from carboxylic acid vinyl esters by addition to dialkyl phosphites (DE-OS 2,127,821):

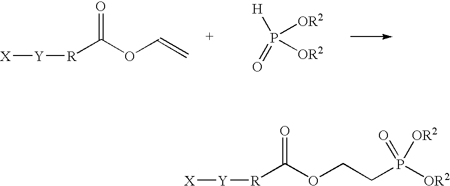

Concrete example:

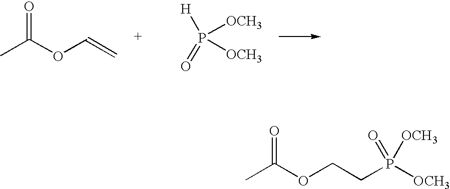

The phosphonic acids of Formula (1) with p=0, n=1, m=1, R=—CH₂—(C═CH₂)—, R²=H, Y²=CO—C, CO—NR⁸ and X=H or Y² is absent and X=COOR⁸, Z², R⁴ and R⁵ are absent (ACPE) can be prepared by reaction of alkylphosphonic acid esters APE (R²=alkyl), which are Z-functionalized at the alkyl radical, with allyl halogenides (U=halogen, above all Cl or Br) and subsequent splitting of the alkyl groups R² accompanied by the use of the methods known in organic chemistry for linking C—C, or C—O bonds (cf. C. Weygand, G. Hilgetag, Organisch-chemische Experimentierkunst [Organic-chemical experimental techniques], Johann Ambrosius Bart Verlag, Leipzig 1970, p. 963f., 362f. and 657f.).

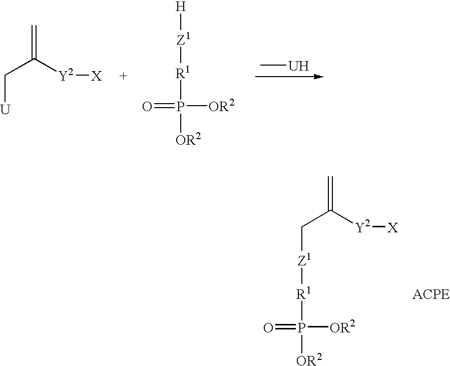

Concrete example:

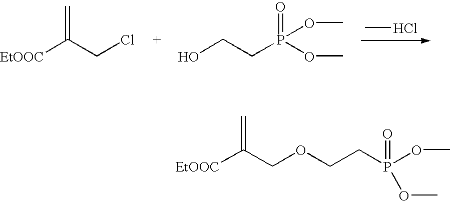

Most of the simple allyl halogenides are commercially available, activated α-halogen methylacrylic compounds can be obtained by reaction of acrylic compounds with formaldehyde in the presence of 1,4-diazabicyclo[2.2.2]octane (DABCO) and subsequent halogenation with inorganic acid chlorides, such as SOCl₂, PCl₃ or PBr₃ (S. C. Warren, L. J. Mathias, J. Polymer Science, Part A: Polymer Chemistry 28 (1990) 1637), e.g.:

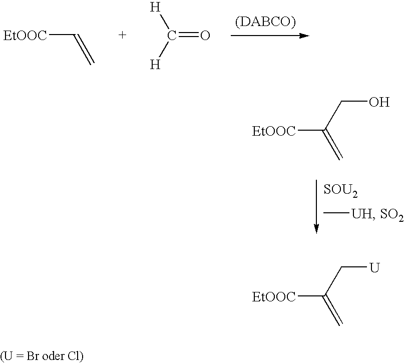

Phosphonic acids of Formula (1) with p=0, n=1, m=1, R=—CH₂—(C═CH₂)—, R²=H, X=CO—W (W=N(R¹²)₂ or OR¹¹) and Y², Z², R⁴ and R⁵ are absent (ACPA) can be prepared by reaction of dialkoxy phosphoryl acrylic acids DPA with amines or alcohols in the presence of a suitable condensing agent and subsequent hydrolysis of the phosphonic acid ester groups.

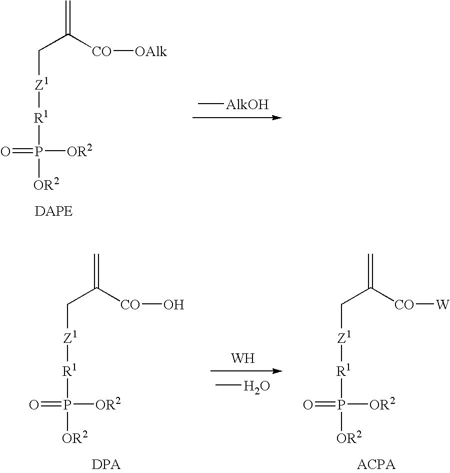

Carbodiimides or phosphoroxychloride (Houben-Weyl, Vol. 15/2, Peptide [Peptides]; 4^{th} edition, Georg Thieme Verlag, Stuttgart 1974, p. 103ff and 232ff) can be used as condensing agents. The dialkoxy phosphoryl acrylic acids DPA used can be prepared from the corresponding dialkoxy phosphoryl acrylic acid alkyl esters DPAE (cf. N. Moszner, F. Zeuner, U. K. Fischer, V. Rheinberger, Macromol. Chem. Phys. 200 (1999) 1062) by selective alkaline hydrolysis.

Concrete example:

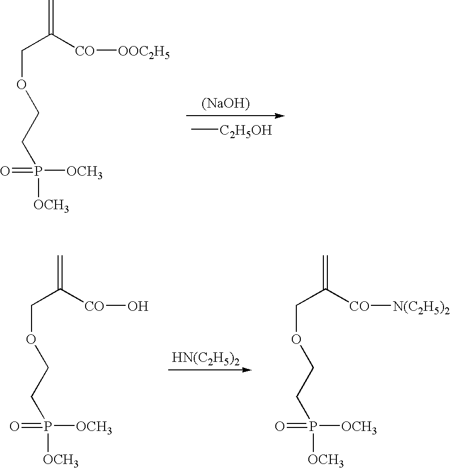

In addition, functionalized alkyl phosphonic acid esters are obtained by acylation of alkyl phosphonic acid esters APE (R²=alkyl), which are functionalized at the radical Z¹, with carboxylic acids:

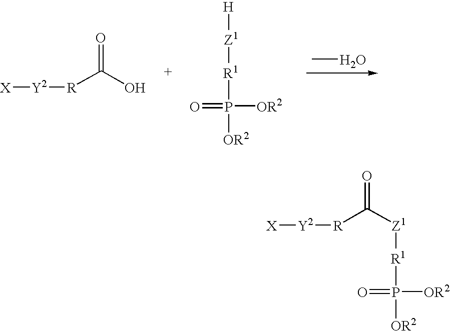

Concrete example:

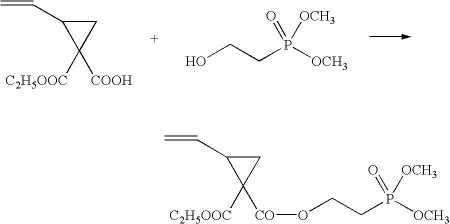

Corresponding vinylcyclopropane-1,1-dicarboxylic-acid monoesters can be prepared by reaction of malonic esters with 1,4-dibromo-but-2-enes and after subsequent hydrolysis of the diester into the monoester (N. Moszner, F. Zeuner, V. Rheinberger, Macromol. Rapid Commun. 18 (1997) 775).

The complete hydrolysis of the phosphonic acid diesters into the corresponding diphosphonic acids takes place favourably by silylation with trialkylsilyl halogenides, e.g. trimethylsilyl chloride/(NaI or NaBr), and subsequent reaction with alcohols or water (S. Freeman, J. Chem. Soc., Perkin Trans. 2, 1991, 263.). Sterically hindered silyl halogenides are used for the selective hydrolysis of only one ester function. If a carboxylic-acid ester function is to be saponified at the same time, hydrolysis is carried out with alkaline lye and after hydrolysis has taken place the phosphonic acids are released again by acidification. The salts are obtained by neutralization of the phosphonic acids with 1 equivalent of the corresponding hydroxide.

Concrete example:

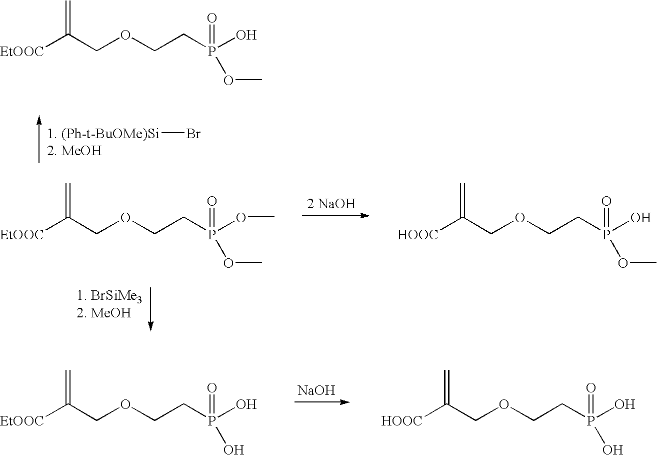

Examples of the phosphonic acids according to the invention of Formula 1 are i.a.:

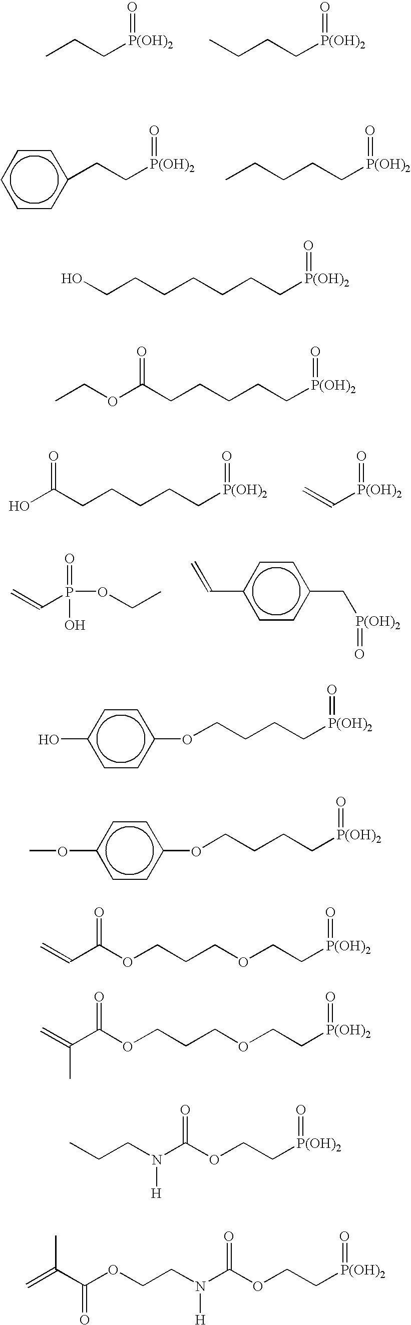

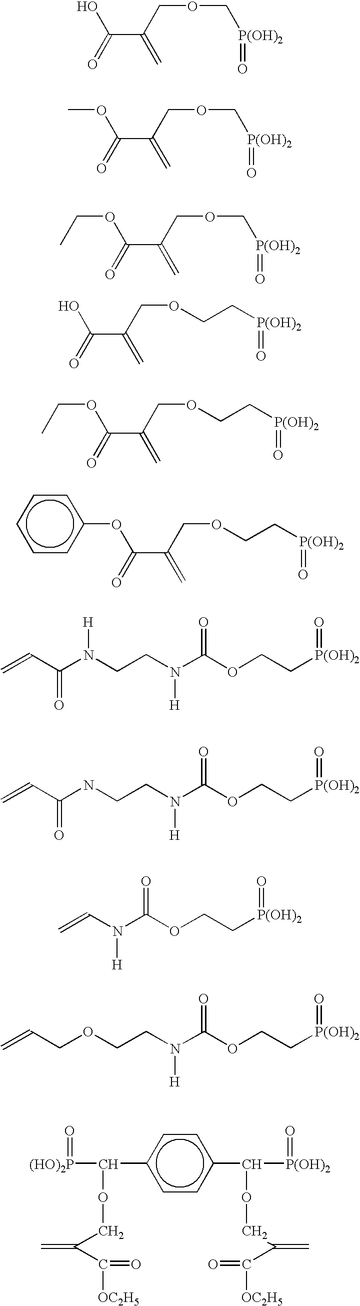

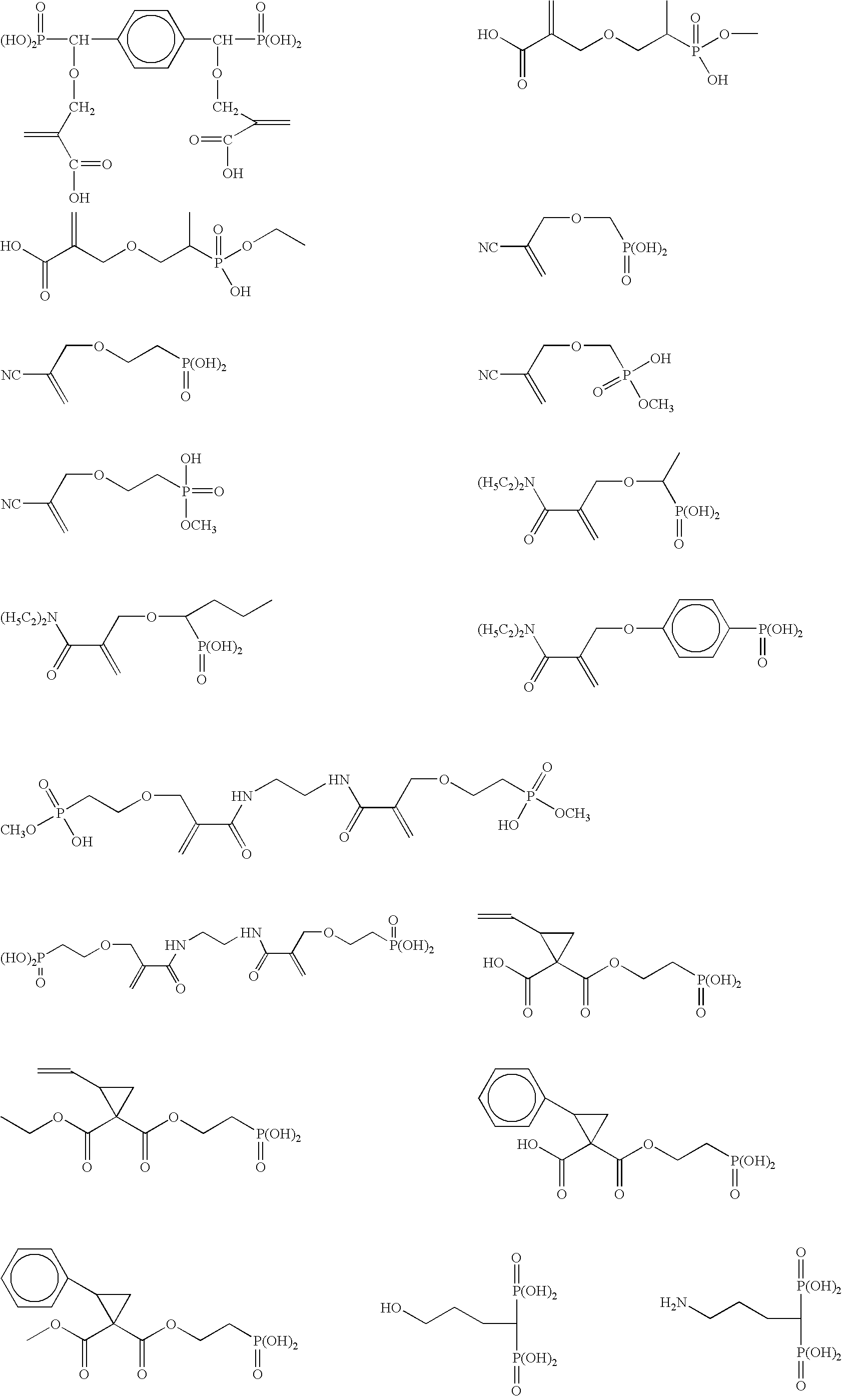

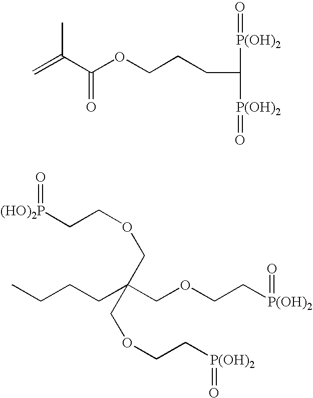

Some suitable phosphonic acids are commercially available, such as e.g. vinyl phosphonic acid (Clariant), 1-hydroxyethane-diphosphonic acid, 2-hydroxyethyl phosphonic acid (Rhodia), or can be prepared in the manner described above.

The compounds disclosed in DE 197 46 708 form a further group of preferred phosphonic acids. These are acids according to Formula 1 wherein

n is 1,

m is 1,

p is 0,

R is a C₁ to C₃ alkylene or phenylene radical,

R² is H,

R⁴ is a branched or straight-chained C₁ to C₆ alkylene radical which can be substituted by 1 to 2 fluorine atoms and/or 1 phenyl radical or is absent,

R⁵ is a 1-methylene ethane-1,2-diyl radical,

Z² is absent,

Y² is O or is absent,

X is COOR¹¹ and

R¹¹ is H or a C₁ to C₅ alkyl or phenyl radical.

Also preferred are the phosphonic acids disclosed in DE 197 46 708 according to Formula 1 in which

n is 2,

m is 2,

p is 1,

R is a quadrivalent aliphatic, aromatic, or aliphatic-aromatic hydrocarbon radical with 2 to 12 carbon atoms,

R¹ is absent,

R² is H,

R³ is a C₁ to C₃ alkylene or phenylene radical or is absent,

R⁴ is a branched or straight-chained C₁ to C₆ alkylene radical which can be substituted by 1 to 2 fluorine atoms and/or 1 phenyl radical or is absent,

R⁵ is a 1-methylene ethane-1,2-diyl radical,

Z¹, Z² are absent,

Y¹ is absent,

Y² is O or is absent,

X is COOR¹¹ and

R¹¹ is H or a C₁ to C₅ alkyl or phenyl radical.

The preparation of these phosphonic acids is described in DE 197 46 708.

Quite particularly preferred phosphonic acids are 2-[4-(dihydroxyphosphoryl)-2-oxa-butyl]-acrylic acid and 2-[4-(methoxy-hydroxyphosphoryl)-2-oxa-butyl]-acrylic acid.

An advantage of the phosphonic acids preferred according to the invention is to be seen in the fact that they have a self-conditioning effect, i.e. abraded dentine and plaque residue need not be removed before the application of the desensitization agents, but do not etch dentine.

In addition to the phosphonic acids, carboxylic acids such as maleic acid and trichloroacetic acid, and in particular sulphonic acids such as sulphosalicylic acid (2-hydroxy-5-sulphobenzoic acid), are suitable for the preparation of compositions.

The named acids can be used alone or as a mixture to prepare the compositions according to the invention. Mixtures which contain 1 to 4, in particular 2 or 3 different acids, are preferred. Mixtures which contain at least one phosphonic acid, quite particularly preferably two or 3 phosphonic acids, are particularly preferred.

Polymers which are soluble in water or water/alcohol mixtures are particularly suitable as organic, carboxyl and/or hydroxyl-group-containing polymers for combination with the acid. Polysaccharides, polyethylene glycols, polyacrylic acids, polyacrylamides, polyvinylpyrrolidines and mixtures of these substances are preferred.

Preferred polysaccharides are chitin, chitosan and glucan.

Preferred polyethylene glycols (PEGs) are those with a molecular weight of 200 to 20,000, particularly preferably 500 to 2,000 and quite particularly preferably approximately 1,000 g/mol. Polyethylene glycol dimethacrylate with a molecular weight of 1,000 g/mol (PEG1000DMA) is quite particularly preferred.

Preferred polyacrylic acids are those with a molecular weight of 10,000 to 60,000 g/mol, particularly preferably 15,000 to 35,000 g/mol. Polyacrylic acid (PAA) with an average molecular weight of approximately 30,000 g/mol, which was modified by reaction with 0.5 mol glycidyl methacrylate (GMA) per acrylic acid component in the polymer (PAA/GMA), is quite particularly preferred. The modified polyacrylic acid has an average molecular weight of 40,000 g/mol.

A mixture of different polymers, particularly preferably a mixture of polyethylene glycol and polyacrylic acid, quite particularly preferably a mixture of the above-defined preferred polyethylene glycols and polyacrylic acids, are preferably used as a carboxyl and/or hydroxyl-group-containing polymer.

Acid and carboxyl and/or hydroxyl-group-containing polymer are preferably used in a weight ratio of acid to polymer of 1:4 to 1:8, preferably 1:5 to 1:6.

For the treatment of hypersensitive teeth, acid and polymer are mixed with each other and applied to the tooth to be treated. The compositions according to the invention are liquid. In addition to acid and polymer, they preferably also contain a solvent suitable for use in the patient's mouth, preferably water, ethanol or a mixture of same. When using solid acids and polymers, the use of a solvent is compulsory. After application, the tooth or teeth are dried for example with an air stream.

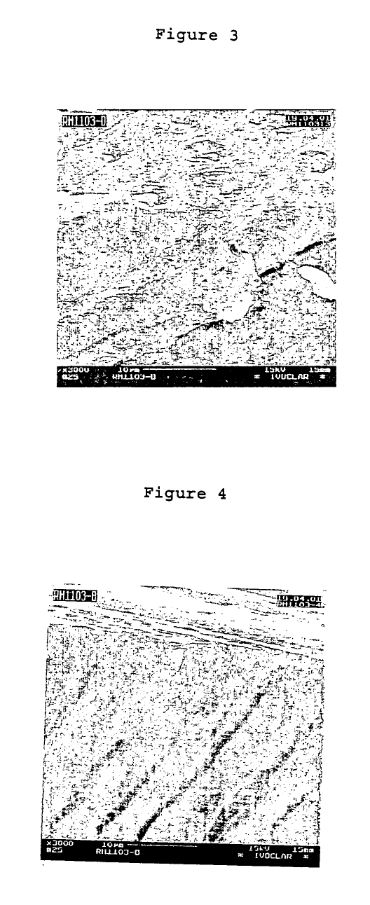

It has been found that the simultaneous use of acid and organic, hydroxyl and/or carboxyl-group-containing polymers causes the dentinal tubules to be practically completely occluded. Through a special imaging technique using an electron microscope, it could be shown that the occlusion is achieved by plugs which extend far into the tubules (FIG. 3) and guarantee a secure and long-lasting protection. This result is surprising since, as a rule, a sensitizing effect is attributed to acids.

In contrast, the preparations for the desensitization of teeth known in the state of the art do not yield any continuous, uniform, plug-shaped precipitates, but merely web-shaped structures (FIG. 4) which, although they represent a certain barrier against movements of the dentinal fluid, cannot guarantee a lasting desensitization of the teeth.

It is assumed that when acids used according to the invention and the polymers used according to the invention meet the dentinal-fluid proteins, simultaneous and possibly mutually dependent precipitation of proteins, calcium and polymer results, which leads to the development of massive plugs which are anchored in the tubules by additional reaction with the Ca portions of the walls of the tubules.

The compositions according to the invention contain no glutaraldehyde and preferably also no hydroxyethyl methacrylate.

In addition to acid and polymer, the compositions according to the invention can contain additional components for the further improvement of their properties.

For example, a rapid primary desensitizing effect can be achieved by a short-term increase of the extracellular potassium-ion concentration through the addition of potassium ion-releasing compounds, preferably KF, KCl, potassium oxalate, K₂SO₄, K₂CO₃, the potassium salts of organic compounds, e.g. of polyacrylic acids and saccharic acids. A similar effect can be achieved by the addition of strontium ions which are added preferably in the form of SrCl₂.

The precipitation of calcium can be favoured by the formation of calcium fluoride through the use of fluoride-ion-releasing compounds, preferably NaF, KF, organic and inorganic amine fluorides, SnF₂ and ZnF₂.

A particularly preferred additive is potassium fluoride which can release both potassium and fluoride ions.

Furthermore, the addition of film-forming substances which effect a mechanical occlusion of the tubules is advantageous. As a result, on the one hand the achievement of a rapid primary effect is favoured, and on the other hand the composition in the tubules is fixed and thus a deep penetration of same into the tubules and the development of a fixed plug is promoted.

Cellulose derivatives, in particular cellulose ethers, such as for example hydroxypropyl cellulose, are preferred as film-forming substances.

The pH value of the compositions according to the invention is preferably in the range of 1 to 4, particularly preferably 1.5 to 3.5 and quite particularly preferably 2 to 3. In order to set and/or keep the pH level constant, it can be advantageous to add suitable buffer systems, such as citrate, phosphate, phosphonate, acetate, carbonate, sulphonate buffers, preferably phosphonate or sulphonate buffers. Within the indicated pH range, a good conditioning of the dentine surface is achieved and at the same time causes protein and calcium precipitation.

Preferred solvents for the preparation of the compositions according to the invention are water and alcohols, such as methanol, isopropanol and in particular ethanol, as well as mixtures of water and alcohol, particularly preferably mixtures of approximately 50 wt.-% water and approximately 50 wt.-% ethanol (based on the overall mass of the solvent). Mixtures of water and alcohol contain preferably at least 20 wt.-% water, based on the solvent mass.

The named components are used preferably in the following quantities which can be chosen independently of each other:

0.5 to 40 wt.-%, preferably 1.0 to 10.0 wt.-% acid,

1.0 to 50 wt.-%, preferably 5 to 35 wt.-% carboxyl and/or hydroxyl-group-containing polymer,

0.5 to 30 wt.-%, preferably 1.0 to 10 wt.-% of a film-forming component,

0.1 to 2.0 wt.-%, preferably 0.1 to 1.0 wt.-% fluoride ions,

0.1 to 10 wt.-%, preferably 0.1 to 5 wt.-% potassium ions,

0 to 97.8 wt.-%, preferably 40 to 80 wt.-% solvent.

Compositions are preferred in which the quantities of all components are within the defined ranges.

The total quantity of carboxyl and/or hydroxyl-group-containing polymer is composed, according to a preferred version, of 1.0 to 40 wt.-%, preferably 2.0 to 10 wt.-% polyacrylic acid and

1.0 to 40 wt.-%, preferably 5.0 to 30 wt.-% polyethylene glycol dimethacrylate.

In addition, the compositions can contain 0.1 to 20 wt.-% SrCl₂.

Moreover, further additives such as gingiva-protecting substances, preferably dexpanthenol, chitosan and hyaluronic acid, and flavourings, for example mint, can be added. Dexpanthenol is preferably used in a quantity of 0 to 5 wt.-%, in particular 0.5 to 2.0 wt.-%, chitosan and hyaluronic acid each in a quantity of 0 to 20 wt.-%, in particular 0 to 5 wt.-%, in each case based on the total mass of the composition. Flavourings are preferably used in a quantity of 0.1 to 1.0 wt.-%.

Compositions which contain

| | |
|---|---|
| 1 to 5 wt. - % | phosphonic acid, in particular one or more |
| | of the above defined preferred phosphonic |
| | acids, |
| 3 to 7 wt. - % | polyacrylic acid, |
| 15 to 25 wt. - % | polyethylene glycol dimethacrylate, |
| 3 to 7 wt. - % | hydroxypropyl cellulose, |
| 0.1 to 1.0 wt. - % | potassium fluoride, |
| 0.05 to 0.2 wt. - % | flavouring and |
| 53.8 to 76.85 wt. - % | ethanol/water mixture (approx. 50 wt. - %). |
| | are quite particularly preferred. |

Unless otherwise stated, all percentages here relate to the total mass of the composition.

As the presence of the acid can have an adverse effect on shelf life, acid, polymer and if present the other components of the compositions according to the invention are sold preferably in spatially separated form, for example in the form of kits. A kit contains e.g. a vessel with acid or a solution of the acid in a suitable solvent, such as water, (acid component) and a second vessel with the polymer and optionally other components, or a solution of the polymer and optionally other components (polymer component). Alternatively, multi-chambered vessels, for example double-chambered vessels, can be used which contain the acid and the other components in separated chambers. The components of the composition can also be divided into more than two vessels or vessel chambers. The compositions of acid component and polymer component are preferably measured such that the above-defined compositions are obtained when the components are combined.

Kits in which the acid is applied to a brush are particularly preferred. To this end, preferably a solid acid is dissolved in a solvent, the solution is applied to a brush and then the solvent is evaporated. Before use, the brush is dipped into a solution of the other components and then the tooth or teeth are treated with this. In this version, the quantity of the solution of the other components is tailored preferably to a single use. Size, shape and bristle material of the brush are chosen preferably such that the brush absorbs the quantity of acid which produces the desired composition together with the second component.

The brush is loaded preferably with a quantity of 2 to 15 mg, particularly preferably 2 to 8 mg, quite particularly preferably 2.5 to 4 mg and quite particularly approximately 3 mg acid per brush.

The quantity of the second component is for example approximately 60 mg for the single use, so that with the acid quantity of the brush a total mass of the composition of 62.5 to 75 mg results. The composition of the second component is chosen such that, after the combination of same with the acid of the brush, the total composition is within the above-defined ranges. Brush and solution are housed preferably in a double-chambered vessel such that brush and fluid can be brought into contact with each other by simply moving the brush. Double-chambered vessels of this type are described e.g. in DE 199 56 705 A1.

The compositions according to the invention are generally suitable for the precipitation of protein, in particular however for the desensitization of sensitive teeth.

Dentinal tubules are frequently also exposed when a dentist operates on teeth, for example when drilling or abrading teeth, which often causes a sensitization. The compositions according to the invention do not impair the effect of customary filling composites and can therefore be combined advantageously with these, i.e. cavities can be treated with a composition according to the invention, for example before the filling is laid, and then provided with the filling.

The invention is described in the following with reference to examples.

### EXAMPLES

### Preparation of Phosphonic Acids

### Example 1

### 2-[4-(dihydroxyphosphoryl)-2-oxa-butyl]-acrylic acid (1)

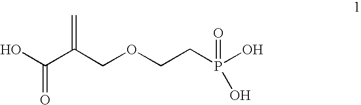

A solution of 69.6 g (1.74 mol) NaOH in 700 ml water was added dropwise to a solution in 50 ml water of 66.6 g (0.28 mol) 2-[4-(dihydroxyphosphoryl)-2-oxa-butyl]-acrylic acid ethyl ester, which can be obtained by reacting 2-hydroxyethyl phosphonic acid diethyl ester with α-chloromethyl acrylic acid ethyl ester and subsequent hydrolysis with trimethyl bromosilane (cf. N. Moszner, F. Zeuner, U. K. Fischer, V. Rheinberger, Macromol. Chem. Phys. 200 (1999) 1062), such that 5° C. is not exceeded. After heating to room temperature, the mixture was stirred for 16 h. The aqueous solution was washed 3 times with 100 ml methylene chloride each time and then set at approximately pH 1 with 20% sulphuric acid (approx. 500 ml, T<10° C.) accompanied by stirring and cooling (ice bath). Precipitated sodium sulphate was filtered off and the solution was washed again with 250 ml methylene chloride. Then the aqueous phase was saturated with common salt and extracted 3 times with 250 ml tetrahydrofuran (THF, stabilized with 300 ppm 2,6-di-tert.-butyl-p-cresol) each time. The combined THF solutions were dried with sodium sulphate and concentrated to dryness. The obtained crystal pulp was dried off firstly in fine vacuum and then finally dried in the dessicator over P₂O₅. 48.8 g (83%) of a white powder is obtained which melts at 119-120° C.

HPLC: >99%

C₆H₁₁O₆P Calc.: C 34.30 H 5.28

(210.12) Found: C 34.85 H 5.29

IR: 463 (m), 505 (w), 533 (w), 675 (w), 738 (w), 785 (w), 825 (w), 969 (s; sh), 1027s), 1100 (s; sh), 1172 (m), 1255 (s), 1278 (s), 1370 (w), 1396 (w), 1396 (w), 1427 (w), 1633 (m), 1694 (s) 2304 (m, b), 2924 (s; b)

¹H-NMR (400 MHz, DMSO-d₆, ppm): 1.82-1.97 (m; 2H; CH₂—P), 3.56-3.65 (m; 2H; OCH₂CH₂), 4.15 (s; 2H; CH₂C═C), 5.81 and 6.14 (s, 2×1H, ═CH₂); 10.29 (s; 3H; OH).

¹³C-NMR (100 MHz; DMSO-d₆, ppm): 27.17 and 28.50 (CH₂P), 65.01 and 67.60 (CH₂OCH₂), 124.43 (CH₂═C), 137.24 (CH₂═C), 167.48 (C═O). ³¹P-NMR (162 MHz, DMSO-d₆, ppm): 23.82

### Example 2

### Step 1: (cis/trans)-1-carboxyethyl-2-vinyl-cyclopropane-1-carboxylic acid-[2-(dimethoxyphosphoryl)ethyl]ester (2)

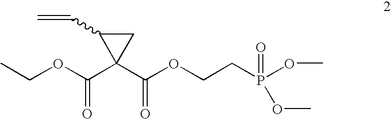

36.8 g (0.2 mol) (cis/trans) -2-vinylcyclopropane-1,1-dicarboxylic acid monoethyl ester, 32 mg hydroquinone monomethyl ether, 732 mg (6.0 mmol) 4-dimethylaminopyridine and 30.8 g (0.2 mmol) (2-hydroxyethyl)-phosphonic acid dimethyl ester were dissolved in 800 ml absolute methylene chloride and cooled to −5° C. 38.4 g (0.2 mol) N-(3-dimethylaminopropyl)-N′-ethyl-carbodiimide-hydrochloride were added in portions to the slightly opaque solution accompanied by stirring. After heating to room temperature, the mixture was stirred for 20 h. The solution was washed twice with 200 ml 2 N hydrochloric acid, saturated sodium hydrogen-carbonate solution and saturated common-salt solution each time and dried over sodium sulphate. The solvent was distilled off and the remaining colourless oil distilled in high vacuum. 32.2 g (50%) of a colourless oil were obtained.

HPLC: 95%

| C₁₃H₂₁O₇P | Calc.: | C 48.75 | H 6.61 |
|---|---|---|---|
| (320.28) | Found: | C 48.90 | H 7.00 |

IR: 2956 (w, CH₂, CH₃), 1720 (s, C═O), 1638 (w, C═C), 1447 (m, CH₂, CH₃), 1370 (m, CH₃), 1021 (ss, C—O—C).

¹H-NMR (400 MHz, CDCl₃ ppm): δ=1.26 (t; J=7.2 Hz, 3 H, CH₂CH₃), 1.57-1.61, 1.71-1.74 (m; 2×1 H, CH₂-cyclopropyl), 2.16-2.25 (m; 2 H, CH₂P), 2.55-2.65 (CH-cyclopropyl), 3.75, 3.78 (s; 2×3 H, OCH₃), 4.20 (q; J=7.0 Hz, OCH₂CH₃), 4.33-4.38 (m; 2 H, OCH₂CH₂P), 5.24 (dd; 2 H, ═CH₂), 5.39-5.49 (m; 1 H, ═CH).

¹³C-NMR (CDCl₃, 100 MHz, ppm): δ=12.8 (CH₃CH₂), 19.1 (CH₂-cyclopropyl), 23.5 (d, J_{C—P}=141 Hz, CH₂P), 30.0 (CH-cyclopropyl), 34.3 (CO—C—CO), 51.1 (OCH₃), 58.1, 60.0 (2×OCH₂), 117.4 (═CH₂), 131.5 (═CH), 165.6, 167.8 (2×C═O).

³¹P-NMR (162 MHz, CDCl₃, ppm): δ=29.3

### Step 2: (cis/trans)-1-carboxyethyl-2-vinyl-cyclopropane-1-carboxylic acid-[2-(dihydroxyphosphoryl)ethyl]ester (3)

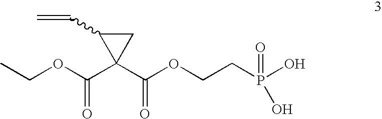

9.61 g (30 mmol) (cis-trans) -1-carboxyethyl-2-vinyl-cyclopropane-1-carboxylic acid-[2-(dimethoxyphosphoryl)ethyl]ester 2 were dissolved in 20 ml absolute methylene chloride and 11.5 g (75 mmol; 9.71 ml) trimethylbromosilane were added dropwise to this solution. The mixture was left to react for 3 h at 40° C. and then the methylene chloride and excess trimethylbromosilane were distilled off. The formed silyl ester was reacted with 35 ml anhydrous methanol and stirred for 6 h at room temperature. The methanol was distilled off again and the residue dried in fine vacuum. For purification, the crude product is taken up in 50 ml water, reacted in portions with 3.15 g (37.5 mmol) sodium hydrogen carbonate and washed twice with 25 ml methylene chloride each time. Then the aqueous phase is acidified with hydrochloric acid accompanied by ice cooling, and the white emulsion, from which the phosphonic acid partially separates, is extracted twice with 25 ml methylene chloride each time. The combined organic phases were dried with sodium sulphate, the methylene chloride distilled off and the residue dried in fine vacuum. 7.50 g (86%) of a highly viscous oil are obtained.

HPLC: 95%

| C₁₁H₁₇O₇P | Calc.: | C 45.20 | H 5.88 |
|---|---|---|---|
| (292.25) | Found: | C 44.98 | H 5.70 |

IR: υ=3200 (sb, OH), 2981 (w, CH₂, CH₃), 1720 (s, C═O), 1637 (m, C═C), 1445 (w, CH₂, CH₃), 1371 (m, CH₃), 1195, 1126 (s, C—O—C), 989, 917 cm⁻¹ (s, ═C—H).

¹H-NMR (CDCl₃, 400 MHz, ppm): δ=1.26 (t; J=7 Hz, 3 H, CH₃), 1.63-1.67, 1.75-1.78 (2 m; 2×1 H, CH₂-cyclopropyl), 2.17-2.25 (m; 2 H, CH₂P), 2.60-2.65 (m; 1 H, CH-cyclopropyl), 4.20 (q; J=7 Hz, 2 H, CH₂CH₃), 4.31-4.51 (m; 2 H, CH₂CH₂P), 5.14-5.16, 5.29-5.34 (m; 2×1 H, ═CH₂), 5.41-5.50 (m; 1 H, ═CH), 10.56 (br.; 2 H, OH).

¹³C-NMR (100 MHz, CDCl₃, ppm): δ=14.2 (CH₃), 20.8 (CH₂-cyclopropyl), 26.2 (d, J_{C—P}=142 Hz, CH₂P), 32.3 (CH-cyclopropyl), 35.6 (CO—C—CO), 59.9, 61.9 (2×OCH₂), 119.1 (═CH₂), 132.6 (═CH), 167.8, 169.2 (2×C═O), 166.9, 169.9 (C═O, cis-isomer)

³¹P-NMR (162 MHz, CDCl₃, ppm): 2 isomers: δ=29.0 (86%)+28.9 (14% cis-isomer)

### Example 3

### Step 1: 5-(dimethoxyphosphoryl)-2-methylene-4-oxa-5-phenylpentanoic acid ethyl ester (4)

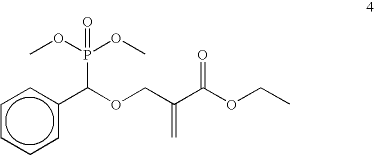

21.6 g (0.1 mol) [(dimethoxyphosphoryl)-hydroxymethyl]benzene, 10.1 g (0.1 mol) triethylamine and 0.02 g phenothiazine were dissolved in 200 ml absolute THF under argon and 14.86 g (0.1 mol) 2-chloromethyl acrylic acid ethyl ester were added dropwise to this solution. The mixture was left to react for 15 h at 60-64° C., cooled to room temperature and the precipitated triethylamine hydrochloride was sucked off. The precipitate was washed with diethyl ether and the washing ether combined with the filtrate. The combined organic solutions were then washed with 400 ml water and the washing water was re-extracted 3 times with 100 ml diethyl ether each time. The organic solutions were combined again, washed with 100 ml saturated common-salt solution and dried over sodium sulphate. After concentration in the rotary evaporator and removal of the remaining THF, 23 g of a colourless liquid crude product were obtained. For further purification, distillation was carried out in high vacuum, accompanied by addition of 0.1 g phenothiazine, and 15.9 g (48%) of a colourless oil were obtained.

| C₁₅H₂₁O₆P | Calc.: | C 54.88 | H 6.45 |
|---|---|---|---|
| (328.30) | Found: | C 54.70 | H 6.23 |

IR: υ=465 (s,b), 701 (m), 771 (w), 832 (m), 1031 (s, sh), 1095 (s), 1181 (s), 1262 (s), 1309 (m), 14001 (w), 1453 (m), 1637 (w), 1718 (s), 2854 (w), 2956 (m).

¹H-NMR (400 MHz, CDCl₃, ppm): 1.29 (t; 3H, CH₃CH₂), 3.65-3.73 (2d; 6 H, CH₃O), 4.14-4.33 (m; 4H, CH₂), 4.78 (d; 1H, CH), 5.97 and 6.34 (s; 2×1H, CH₂═), 7.34-7.47 (m; 5H, CH-aromatic).

¹³C-NMR (100 MHz, CDCl₃, ppm): 14.5 (s, CH₃—CH₂); 53.8 and 54.2 (s, 2×CH₃O), 61.1 (OCH₂CH₃), 69.88 (s, OCH₂C═CH₂), 78.5 (d, J_{C—P}=168 Hz, CHP); 126.9 (s, CH₂═); 128.3 and 128.9 (s, CH— aromatic); 135.0 and 136.8 (CH₂═C and C-aromatic), 165.4 (C═O).

³¹P (162 MHz, CDCl₃, ppm): 21.30 (s)

### Step 2: 5-(dihydroxyphosphoryl)-2-methylene-4-oxa-5-phenylpentanoic acid ethyl ester (5)

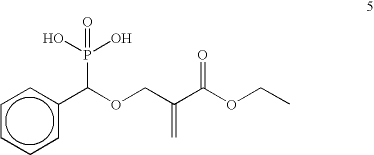

14.4 g (0.044 mol) 5-(dimethoxyphosphoryl)-2-methylene-4-oxa-5-phenylpentanoic acid ethyl ester 4 were reacted and worked up analogously to Example 2 (stage 2) with 17.2 g (0.11 mol) trimethylsilyl bromide in 40 ml methylene chloride. 10.0 g (76%) of a white powder were obtained.

| C₁₃H₁₇O₆P | Calc.: | C 52.00 | H 5.71 |
|---|---|---|---|
| (300.25) | Found: | C 52.15 | H 5.67 |

IR: υ=698 (s), 739 (w), 805 (w), 819 (w), 970 (s), 1026 (s, sh), 1094 (s, sh), 1178 (s, sh), 1280 (s, sh), 1320 (m), 1340 (m), 1402 (m, sh), 1453 (m, sh), 1490 (m), 1634 (m), 1713 (s), 2321 (m), 2910 (m), 2949 (m), 2982 (m).

¹H-NMR (400 MHz, CDCl₃, ppm): 1.22 (t; 3H, CH₃), 4.06-4.17 (m, 4H, CH₂), 4.50 (d; J=4 Hz, 2H, CHP), 5.89 and 6.19 (s, 2×1H, CH₂═), 7.18-7.33 (m, 5H, CH-aromatic); 10.79 (s; 2H, OH, H/D exchange).

¹³C-NMR (100 MHz, CDCl₃, ppm): 14.1 (CH₃), 60.7 (OCH₂CH₃, 68.5 (CH₂OCH), 78.0 (d, J_{C—P}=166.5 Hz, CH—P), 127.5-128.3, 135.2, 136.2 (all C-aromatic+CH₂═C), 166.1 (C═O).

³¹P-NMR (162 MHz, CDCl₃, ppm): 20.1.

### Preparation and Examination of Desensitization Agents

### Example 4

### Preparation of a Single-component Desensitization Agent

A desensitization agent of the following composition was prepared by mixing the components:

| Component | Proportion [wt. - %] |
|---|---|
| Phosphonic acid from Ex. 1 | 4.0 |
| Polyacrylic acid (PAA/GMA) | 5.0 |
| Polyethylene glycol | 20 |
| (PEG 1000 DMA) | |
| Hydroxypropyl cellulose | 5.0 |
| Potassium fluoride | 0.3 |
| Flavouring (Optamint) | 0.1 |
| Ethanol/water (50:50) | 65.6 |

### Example 5

### Preparation of a Two-component Desensitization Agent

A 20 wt.-% solution of phosphonic acid from Example 1 was prepared in ethanol. Small brushes were dipped into this solution and then dried. This process was repeated until the acid quantity was 3.0±0.3 mg per brush after drying.

A mixture of the following composition was prepared as a second component:

| Component | Proportion [g] |
|---|---|
| Polyacrylic acid (PAA/GMA) | 5.0 |
| Polyethylene glycol | 20 |
| (PEG 1000 DMA) | |
| Hydroxypropyl cellulose | 5.0 |
| Potassium fluoride | 0.3 |
| Flavouring (Optamint) | 0.1 |
| Ethanol/water (50:50) | 65.6 |

The second component was divided into portions of 60 mg each. Then in each case a brush was dipped into a 60 mg portion of the second component and mixed thoroughly with the brush. A theoretical acid concentration of approximately 4.8 wt.-% is achieved in the composition.

### Example 6

### Measurement of the Protein Precipitation

In order to determine the protein-precipitating properties of the compositions according to the invention, horse serum was used (horse serum, PAA Laboratories GmbH, Linz, Austria, cat. no. B15-021) which was diluted with physiological common-salt solution (8.5 g NaCl per 1 l H₂O) (1 part by volume horse serum+2 parts by volume common-salt solution), in order to obtain a protein concentration comparable with dentinal fluid. Furthermore, CaCl₂ (2 mM) was added to the diluted protein solution in order to adjust the calcium-ion concentration to that of dentinal fluid.

500 μl of the protein solution was then reacted with 500 μm of the composition to be tested and the mixture was left to stand for 30 minutes at room temperature, centrifuged and the obtained precipitates dried at 75° C. and weighed.

For protein precipitation, the solution (2^{nd} component) described in Example 5 was used which was reacted with phosphonic acid in concentrations of 10 mM to 200 mM (cf. Table 1). 5 precipitations per concentration were carried out and then the average of the determinations was calculated. The results are indicated in Table 1.

These results show that, in the case of the compositions according to the invention, even very small acid concentrations induce a significant protein precipitation, even at 150 mM acid the precipitation is quantitative.

Further compositions according to the invention were tested in the above-described manner. The compositions of the samples and the results are summarized in Table 2.

TABLE 1
Influence of the acid concentration on the protein precipitation

| Acid concentration | Precipitate¹ |
|---|---|
| [mM] | [mg] |
| 10 | 55.3 |
| 25 | 58.2 |
| 50 | 58.8 |
| 75 | 69.1 |
| 100 | 70.8 |
| 150 | 74.2 |
| 200 | 72.3 |

¹Average of 5 measurements

TABLE 2
Protein precipitation of the compositions according to the invention

| | Compostion | | | | | | | |
|---|---|---|---|---|---|---|---|---|
| | (figures in wt. − %, difference relative to 100%:H₂O) | | | | | | | |
| Sam- | | | Polymer | | | | | |
| ple | Acid | | PEG 1000- | PO- | | | | Pellet |
| no. | P acid¹ | SSA² | DMA³ | 25⁴ | KF⁵ | HPC⁶ | EtOH | [mg] |
| 1 | 1.0 | — | 9.0 | — | — | — | — | 24.9 |
| 2 | 1.8 | — | 5.5 | 9.1 | — | — | 4.5 | 42.8 |
| 3 | 2.0 | — | 8.9 | 2.5 | — | — | 0.05 | 57.5 |
| 4 | 1.0 | — | 8.9 | 2.5 | — | 0.25 | 2.15 | 59.3 |
| 5 | — | 2.5 | — | 3.0 | — | — | — | 45.2 |
| 6 | — | 1.4 | 5.5 | 1.4 | — | — | 18.2 | 31.9 |
| 7 | — | 1.3 | 8.9 | 2.5 | 0.05 | — | — | 55.7 |
| 8 | — | 0.6 | 8.9 | 2.5 | 0.05 | 0.25 | 2.1 | 57.4 |

¹Phosphonic acid from Example 1
²Sulphosalicylic acid
³Polyethylene glycol dimethacrylate, molecular weight 1000 g/mol
⁴Polyacrylic acid, mean average molecular weight 40,000 g/mol, modified with 0.5 mol glycidyl methacrylate/mol polyacrylic acid
⁵Potassium fluoride
⁶Hydroxypropyl cellulose

### Example 7

### Electron-microscopical Examination of the Effect of Desensitization Agents on Bovine Teeth

In order to examine the effect of desensitization agents on natural teeth, they were applied to bovine teeth. To this end, the teeth were embedded in synthetic resin and ground to the uppermost dentine layer with an abrasive disk with silicon-carbide abrasive paper (Grit 120/1000). The teeth were then broken out of the synthetic resin and rinsed off with water. Thereafter, the pulp chamber was sealed with a dental varnish customary in the trade (Heliobond, Ivoclar Vivadent AG). The teeth were then subjected to an acid etching by treating the dentine for 15 seconds with a dental etching gel (37% phosphoric acid, Email Preparator Blue, Ivoclar Vivadent AG) customary in the trade. After rinsing with water and drying, the composition to be examined was applied and left on the teeth for 30 seconds. Thereafter the teeth were rinsed again with water, then stored for 6 hours at 37° C. in 0.85% NaCl/2 mM CaCl₂ solution and then dried for 4 days at 75° C.

In order to examine the teeth using an electron microscope, they were broken and both the dentine surface and the fracture edges were examined.

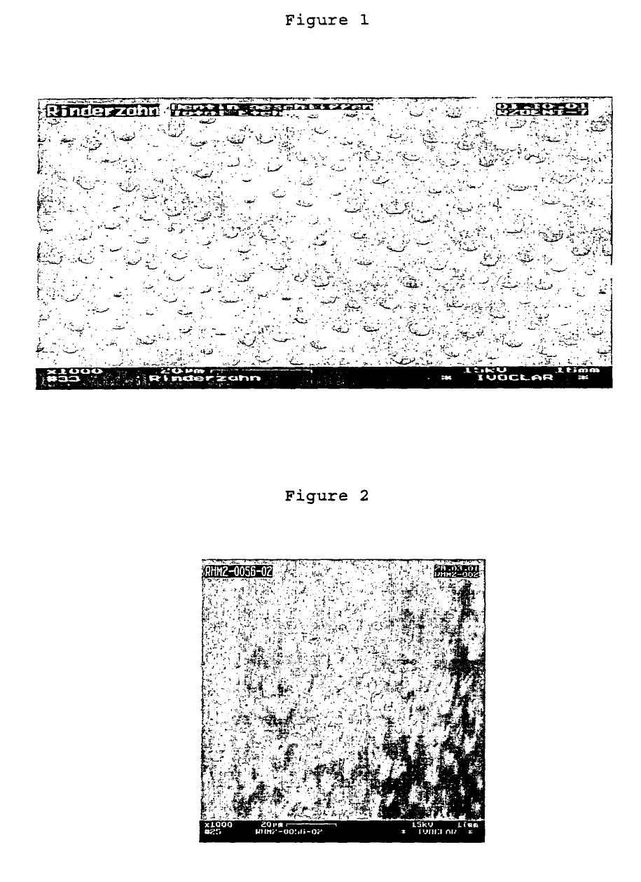

FIG. 1 shows the surface of a ground and etched tooth which was not treated with a desensitization agent. The dentinal tubules are clearly evident.

FIG. 2 shows the surface of a tooth which was treated with the desensitization agent described in Example 4. The magnification corresponds to that of FIG. 1 (×1000). The dentinal tubules are occluded here. The cracks visible in the tubule plugs are to be attributed to the extreme drying of the teeth necessary for the SEM images.

FIG. 3 is an image along the fracture edge of a tooth (×3000). Both the tooth surface with the occluded tubule openings and the cracked tubules are clearly visible. The fracture technique enables a view of the tubules and shows that the occlusion plugs extend far into the tubules, i.e. at least approximately 20 μm. The cracks between the plugs and the tubule wall are again to be attributed to the drying of the teeth and the shrinkage associated with this.

By way of comparison, a tooth was treated in the above-described manner with a desensitization agent according to the state of the art (Gluma, Heraeus Kulzer). The electron-microscope image of the fracture edge of the tooth (magnification of ×3000) shown in FIG. 4 clearly reveals the differences compared with the products according to the invention. Unlike in the case of the compositions according to the invention, no continuous, uniform precipitates are formed, but merely cross-linkages and short plugs.

### Example 8

### Examination of the Effect of Desensitization Agents on Human Teeth Through Laser-scanning Microscopy

The two-component material described in Example 5 was used to treat human molars. In order to facilitate an examination of the teeth through laser-scanning microscopy, a small quantity of fluorescein was first taken up by a brush and mixed with the fluid component of the desensitization agent by multiple dipping (maximum of six times). Immediately afterwards, the product was applied to roughly cleaned human molars and these were then rinsed with water. Thereafter, a slice was cut out of each tooth with a slow-moving saw and examined with a confocal laser-scanning microscope.

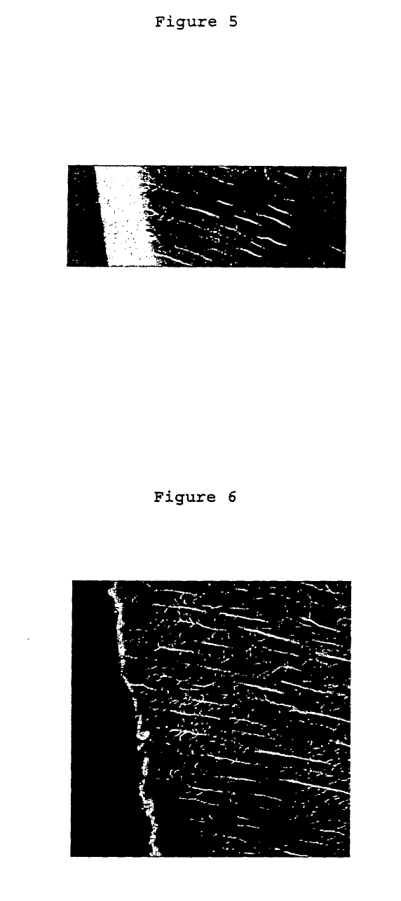

FIG. 5 shows an image of such a slice. The green edge clearly shows film formation both on the dentine and on the enamel of the tooth. The green lines show a deep penetration by the desensitization agent into the tubules. A penetration of more than 200 μm was observed.

In order to simulate the natural pressure of dentinal fluid, in a second experiment the horse-serum solution described in Example 6 was guided through the pulp chamber into the tubules and the desensitization agent applied under a hydrostatic pressure of the simulated dentinal fluid of 60 cm fluid column. It could be proved that the desensitization agent still penetrates 40 to 50 μm into the tubules even under these conditions (FIG. 6, areas with desensitization agent appear green, areas without desensitization agent red).

### Example 9

### Combination of Desensitization Agents with Filling Composites

As a rule, the laying of fillings initially requires a removal of diseased tooth substance, which involves an exposure of dentinal tubules and a sensitization of the tooth. It was therefore examined to what extent desensitization agents according to the invention are compatible with conventional filling composites.

Bovine teeth were initially etched for 15 seconds with an etching gel (37% phosphoric acid, Email Preparator Blue, Ivoclar Vivadent AG) customary in the trade and then rinsed off with water. The desensitization agent described in Example 4 was then applied (for 5 seconds) and left to react for 30 seconds. After further rinsing with water, drying was carried out in the air stream, an adhesion promoter customary in the trade (Excite, Ivoclar Vivadent AG) was applied, left to react for 10 seconds, dried again in the air stream and the tooth was then lit with light of a wavelength from 400 to 510 nm (halogen-light polymerization device, Astralis 7 type, Ivoclar Vivadent AG) at 750 mW/cm² for 20 seconds. Finally, 2 layers of a filling composite customary in the trade (Tetric Ceram, Ivoclar Vivadent AG) were applied and cured in each case by lighting for 40 seconds as described above.

The adhesive strength is then measured in the customary manner. This was 30 MPa while, in the case of teeth treated in the same way without desensitization agent, a value of 29.5 MPa resulted. This difference is statistically insignificant. The desensitization agent thus does not represent an impairment of the adhesion process.

1. Compositions characterized in that they contain an acid and an organic polymer which has carboxyl and/or hydroxyl groups.

2. Composition according to claim 1, characterized in that it contains an acid which has a solubility of 0.5 to 20 wt.-% in water or in a mixture of 50 wt.-% water and 50 wt.-% ethanol.

3. Composition according to claim 1 or 2, characterized in that it contains an acid with protein- and/or calcium-precipitating properties.

4. Composition according to one of claims 1 to 3, characterized in that it contains as an acid a carboxylic acid, sulphonic acid and/or phosphonic acid.

5. Composition according to claim 4, characterized in that it contains a phosphonic acid of formula 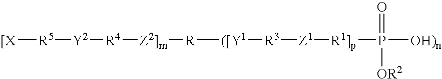
in which
n is 1, 2, 3 or 4,
m is 0, 1 or 2,
p is 0 or 1,
R is a straight-chained or branched aliphatic hydrocarbon radical with 1 to 12 carbon atoms or an aromatic hydrocarbon radical with 6 to 12 carbon atoms or an aliphatic/aromatic hydrocarbon radical with 7 to 16 carbon atoms, which can be substituted by OH, NH₂ and/or COOR⁶,
R¹ is a C₁ to C₁₂ alkylene, C₄ to C₁₂ cycloalkylene, C₆ to C₁₂ arylene or C₇ to C₁₆ alkylenearylene radical, which can be substituted by OH, NH₂ and/or COOR⁶, or is absent,
R² is H, a C₁ to C₆ alkyl or a phenyl radical,
R³, R⁴ each mean, independently of each other, a C₁ to C₁₂ alkylene, C₆ to C₁₂ arylene or C₇ to C₁₆ alkylenearylene radical, which can be substituted by methyl, phenyl or fluorine, or are absent,
R⁵ is —CH═CR¹³—, a prop-1-ene-1,3-diyl, C₁ to C₆ alkenylene, C₃ to C₉ cycloalkylene, C₁ to C₆ alkylene or phenylene radical or a group of formula 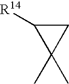
R⁶ is H, a C₁ to C₆ alkyl or a phenyl radical,
Z¹, Z² each mean, independently of each other, CO—O, CO—NR⁷, O—CO—NH, O, NH, S or are absent,
Y¹, Y² each mean, independently of each other, O, CO—O, CO—NR⁸, O—CO—NH or are absent,
R⁷, R⁸ each mean, independently of each other, H, or a C₁ to C₆ alkyl radical,
X is H, CN, N(R⁹)₂, OR¹⁰, COOR¹¹ or CONR₂¹²,
R⁹, R¹⁰, R¹¹, R¹² each mean, independently of each other, H, a C₁ to C₁₀ alkyl or a phenyl radical,
R¹³ is H or a methyl radical,
R¹⁴ is H or a C₁ to C₁₀ alkyl, vinyl or phenyl radical.

6. Composition according to claim 5, characterized in that
n is 1 or 2 and/or
m is 1 and/or
p is 0 and/or
R is an aliphatic straight-chained or branched mono- to pentavalent alkane radical with 1 to 7 carbon atoms, an aromatic hydrocarbon radical with 6 carbon atoms or an aliphatic/aromatic hydrocarbon radical with 8 carbon atoms and/or
R¹ is a methylene or ethylene radical or is absent and/or
R² is H, a methyl or ethyl radical and/or
R³, R⁴ each mean, independently of each other, a methylene, ethylene, trimethylene, p-phenylene, ethylidene, 1-methylene ethane-1,2-diyl radical or are absent and/or
R⁵ is a methylene, ethylene, trimethylene, ethene-1,2-diyl, methylethylene, prop-1-ene-1,3-diyl, or a cyclopropylidene radical monosubstituted in 2 position or is absent and/or
R⁶ is H and/or
Z¹, Z² each mean, independently of each other, CO—O, O—CO—NH or O or are absent and/or
Y¹, Y² each mean, independently of each other, O, CO—O or CO—NR⁸ or are absent and/or
R⁷, R⁸each mean, independently of each other, H or a methyl or ethyl radical and/or
X is H, CN, COOR¹¹ or CONR₂¹² and/or
R⁹, R¹⁰, R¹¹, R¹² each mean, independently of each other, H or a methyl, ethyl or phenyl radical and/or
R¹³ is H or a methyl radical,
R¹⁴ is H or a vinyl or phenyl radical.

7. Composition according to claim 5, characterized in that
n is 1,
m is 1,
p is 0,
R is a C₁ to C₃ alkylene or phenylene radical,
R² is H,
R⁴ is a branched or straight-chained C₁ to C₆ alkylene radical which can be substituted by 1 to 2 fluorine atoms and/or 1 phenyl radical or is absent,
R⁵ is a 1-methylene ethane-1,2-diyl radical,
Z² is absent,
Y² is O or is absent,
X is COOR¹¹ and
R¹¹ is H or a C₁ to C₅ alkyl or phenyl radical.

8. Composition according to claim 5, characterized in that
n is 2,
m is 2,
p is 1,
R is a quadrivalent aliphatic, aromatic, or aliphatic-aromatic hydrocarbon radical with 2 to 12 carbon atoms,
R¹ is absent,
R² is H,
R³ is a C₁ to C₃ alkylene or phenylene radical or is absent,
R⁴ is a branched or straight-chained C₁ to C₆ alkylene radical which can be substituted by 1 to 2 fluorine atoms and/or 1 phenyl radical or is absent,
R⁵ is a 1-methylene ethane-1,2-diyl radical,
Z¹, Z² are absent,
Y¹ is absent,
Y² is O or is absent,
X is COOR¹¹ and
R¹¹ is H or a C₁ to C₅ alkyl or phenyl radical.

9. Composition according to one of claims 4 to 8, characterized in that it contains as carboxylic acid maleic acid and/or trichloroacetic acid.

10. Composition according to one of claims 4 to 9, characterized in that it contains as sulphonic acid sulphosalicylic acid (2-hydroxy-5-sulphobenzoic acid).

11. Composition according to one of claims 1 to 10, characterized in that it contains 1 to 4 different acids.

12. Composition according to one of claims 1 to 11, characterized in that it contains as a polymer a polysaccharide, a polyethylene glycol, a polyacrylic acid, a polyacrylamide, a polyvinylpyrrolidine or a mixture of these substances.

13. Composition according to claim 12, characterized in that it contains as a polymer a mixture of polyethylene glycol dimethacrylate and polyacrylic acid.

14. Composition according to one of claims 1 to 13, characterized in that it also contains fluoride ions.

15. Composition according to one of claims 1 to 14, characterized in that it also contains a potassium ion-releasing compound.

16. Composition according to one of claims 1 to 15, characterized in that it also contains a film-forming component.

17. Composition according to claim 16, characterized in that it contains hydroxypropyl cellulose.

18. Composition according to one of claims 1 to 17, characterized in that it contains

| | |
|---|---|
| 0.5 to 40 wt. - % | phosphonic acid and/or |
| 1.0 to 40 wt. - % | carboxyl and/or hydroxyl-group- |
| | containing polymer and/or |
| 0.5 to 30 wt. - % | of a film-forming component and/or |
| 0.1 to 1.0 wt. - % | fluoride ions and/or |
| 0.1 to 10 wt. - % | potassium ions and |
| 0 to 97.8 wt. - % | solvent. |

19. Composition according to claim 18, characterized in that it contains additionally 0.1 to 1.0 wt.-% flavourings.

20. Composition according to claim 18 or 19, characterized in that it contains as a solvent a mixture of ethanol and water.

21. Composition according to one of claims 18 to 20, characterized in that it contains

| | |
|---|---|
| 1 to 5 wt. - % | of at least one phosphonic acid, |
| 3 to 7 wt. - % | polyacrylic acid, |
| 15 to 25 wt. - % | polyethylene glycol dimethacrylate, |
| 3 to 7 wt. - % | hydroxypropyl cellulose, |
| 0.1 to 1.0 wt. - % | potassium fluoride, |
| 0.05 to 0.2 wt. - % | flavouring and |
| 53.8 to 76.9 wt. - % | ethanol/water mixture (approx. 50 wt. - %). |

22. Kit containing an acid and in spatially separated form thereof an organic, carboxyl and/or hydroxyl-group-containing polymer.

23. Kit according to claim 22, characterized in that the acid is applied to a brush.

24. Kit according to claim 22 or 23, characterized in that it contains a solution of the polymer, the composition of which is measured such that, when the solution is combined with the acid of the kit, a composition according to one of claims 18 to 21 is obtained.

25. Kit according to one of claims 22 to 24, characterized in that acid and polymer are housed in different chambers of a double-chambered vessel.

26. Use of a composition as defined in claims 1 to 21 for the precipitation of protein.

27. Use of a composition as defined in claims 1 to 21 for the desensitization of teeth.

28. Use of a composition according to one of claims 1 to 21 for the preparation of an agent for the desensitization of teeth.
